(19) United States
(12) Patent Application Publication
(10) Pub. No.: US 2011/0300085 A1
(43) Pub. Date: Dec. 8, 2011

(54) Home And Personal Care Compositions

(76) Inventors: Emilie Courbon, Conde Sur Escaut (FR); Lorry Deklippel, Pieton (BE); Tatiana Dimitrova, Braine-L'Alleud (BE); Anne-Lise Girboux, Jurbise (BE); Sophie Hanssens, Chastre (BE); Stephane Ugazio, Soignies (BE)

(21) Appl. No.: 13/132,729

(22) PCT Filed: Oct. 30, 2009
(86) PCT No.: PCT/US09/62704

§ 371 Date: Aug. 29, 2011

(30) Foreign Application Priority Data
Dec. 15, 2008 (GB) 0822823.1

(51) Int. Cl. A61K 8/97; A61K 8/30; A61K 8/36; A61K 8/72; C12N 9/00; C12N 9/42; C12N 9/26; C12N 9/48; A01N 37/36; A01N 31/14; A61K 8/18; A61Q 5/00; A61Q 5/02; A61Q 5/12; A61Q 19/00; A61Q 19/04; A01P 1/00; C11D 3/60; A61K 8/00

(52) U.S. Cl. 424/60; 424/59; 424/65; 424/70.1; 424/74; 424/70.9; 514/738; 424/70.27; 424/70.31; 424/70.22; 424/70.24; 424/729; 424/727; 424/764; 424/750; 424/725; 424/736; 424/735; 424/751; 424/757; 424/758; 424/755; 424/761; 424/777; 424/778; 424/78.03; 435/183; 435/209; 435/201; 435/212; 514/159; 514/717; 424/61; 510/347; 510/343; 510/515; 510/516; 510/517; 510/299

(57) ABSTRACT

The present invention relates to home and personal care compositions comprising a silicone oil-in-water emulsion stabilized by a protein or peptide and at least one home and/or personal care ingredient. Such compositions tend to provide desirable characteristics for the care and conditioning of hair, skin and fabric.

### CROSS-REFERENCE TO RELATED APPLICATIONS

The present application claims priority from GB Patent application 0822823.1 filed on Dec. 15, 2008, the disclosure of which is hereby incorporated by reference.

### BACKGROUND OF THE INVENTION

This invention relates to home and personal care compositions comprising silicone oil-in-water emulsions comprising droplets of silicone oil dispersed in a continuous aqueous phase. Silicones (organopolysiloxanes) are present in many hair care products to enhance the shine and healthy appearance of the hair and are also present in skin care products to enhance the smooth feel of the skin. Silicones are present in laundry products such as rinse cycle fabric softeners to give a soft feel to fabrics.

More and more end users are developing allergies and therefore need products that do not contain potentially irritant and potentially sensitizing molecules as surfactants. This is particularly true for preparations designed for young children, such as baby wipes or baby shampoo. Amphiphilic surfactants may also generate a lot of foam when disposed of in sewage, and are unfriendly to aquatic life. In some applications such as food, cosmetics and household the formulation freedom and choice of surfactants are limited by legislation.

### BRIEF SUMMARY OF THE INVENTION

The present invention relates to home and personal care compositions comprising a silicone oil-in-water emulsion stabilized by a protein or peptide and at least one home and/or personal care ingredient. Such compositions tend to provide desirable characteristics for the care and conditioning of hair, skin and fabric.

### DETAILED DESCRPTION OF THE INVENTION

The home and personal care compositions comprising the silicone oil-in-water emulsion stabilized by a protein or peptide and at least one home and/or personal care ingredient are further described as follows.

The silicone oil phase of the silicone oil-in-water emulsion stabilized by a protein or peptide generally comprises a fluid organopolysiloxane composition. The fluid organopolysiloxane composition may for example have a bulk viscosity of at least 1 or 5 mm²/s up to 1 or even 20 m²/s. The fluid organopolysiloxane may for example be a substantially linear polydiorganosiloxane, for example of viscosity 100 to 60000 mm²/s such as polydimethylsiloxane although branched and/or cyclic polysiloxanes may also be emulsified. The organopolysiloxane fluid may be a non-reactive fluid, for example a linear polydimethylsiloxane tipped with trimethylsiloxy units, or may be a reactive organopolysiloxane fluid.

The silicone oil phase may be a mixture of two or more organopolysiloxanes. For example the silicone oil phase may be a solution of a solid organopolysiloxane gum or resin, or of a highly viscous organopolysiloxane gum, in a low viscosity organopolysiloxane fluid. The organopolysiloxane gum may for example have a viscosity of above 1000 cm²/s or even above 100000 cm²/s. The low viscosity organopolysiloxane fluid may for example have a viscosity in the range 1 to 1000 mm2/s (1 to 1000 mm²/s). The low viscosity organopolysiloxane fluid may be a cyclic polydiorganosiloxane such as decamethylcyclopentasiloxane and/or a linear polydiorganosiloxane such as a linear polydimethylsiloxane tipped with trimethylsiloxy units.

A reactive organopolysiloxane fluid may for example contain reactive groups such as hydroxyl (either Si—OH or alcohol groups), amino, vinyl or Si—H groups. The reactive organopolysiloxane fluid may for example be a silanol-terminated polydimethylsiloxane. The reactive organopolysiloxane fluid may be mixed with a non-reactive organopolysiloxane fluid. An amino-functional organopolysiloxane is alternatively mixed with at least 30%, alternatively at least 50%, of a non-reactive organopolysiloxane fluid such as a linear polydimethylsiloxane tipped with trimethylsiloxy units, based on the weight of the amino-functional organopolysiloxane.

The silicone oil phase is generally present at≧3% by weight of the emulsion and is usually present at≧10 or 20% to allow efficient distribution of the silicone emulsion. The silicone oil phase may form up to 90% by weight of the emulsion, alternatively 85 or alternatively 86%. Typical emulsions for easy incorporation into personal care products may contain 25 to 65% by weight silicone oil phase, alternatively 45 to 55% silicone oil phase.

A wide range of proteins and peptides are effective in stabilizing the silicone oil-in water-emulsion. The protein may for example be a milk-derived protein such as casein or whey protein. Vegetable proteins, in particular cereal proteins such as wheat protein (gluten) have also been found effective. Proteins derived from nuts such as almond or from other pulses, or soy protein, may also be effective. The protein may be in the form of a derivative such as a salt, for example casein may be in the form of sodium caseinate. A suitable sodium caseinate is sold under the trade mark ‘Lactalis’. The protein may be partially hydrolysed. Milk proteins are highly effective emulsion stabilizers without hydrolysis but vegetable proteins may be more effective in partially hydrolysed form. Examples of such hydrolysed proteins are partially hydrolysed gluten products sold by Tate & Lyle under the trade marks ‘Meripro 705’ and ‘Meripro 711’, and an almond-extracted protein hydrolysate sold by Cognis under the trade mark ‘Gluadin Almond’.

Not all proteins and peptides are effective in stabilizing the silicone oil-in-water emulsion. Proteins whose natural function is as structural proteins, such as keratin, the main structural protein of hair and wool, are generally not effective in emulsifying silicones. Proteins from plants—where cellulose, not protein, is the main natural structural material usually effect emulsification of the silicone, as do proteins from natural emulsions such as milk proteins. The effectiveness of a candidate protein may be tested by mixing equal amounts of dimethicone and a 2% aqueous solution of the protein in a high shear mixer and observing whether an emulsion is formed or the dimethicone separates from the aqueous phase after mixing. If an emulsion is formed, and the mean particle size as well as 90th percentile of the distribution are maintained for at least 2 weeks, the protein is effective, although improvements in the particle size and stability of the emulsion may probably be achieved by varying the proportions of materials or using a more sophisticated emulsifying apparatus.

Even the proteins which are effective in emulsifying the silicone may vary in the extent of stabilization of the emulsion over time. For example, the wheat protein hydrolysates ‘Meripro 705’ and the more extensively hydrolysed ‘Meripro 711’ both emulsify the silicone but the emulsions formed using ‘Meripro 705’ are more stable to long term storage. The emulsions formed using ‘Meripro 705’ are also more stable to long term storage than emulsions formed using ‘Gluadin Almond’. The silicone-in-oil emulsions formed using milk proteins such as casein generally have good long term stability.

Mixtures of proteins may be used to stabilize the emulsion, for example a mixture of proteins from different sources such as a milk protein with a vegetable protein or partially hydrolysed vegetable protein.

The amount of protein in the emulsion is generally≧0.25% by weight of the emulsion to achieve stabilization of the emulsion and is alternatively≧0.5 or 0.75%. The amount of protein in the emulsion may be up to 20% by weight of the emulsion, and protein concentrations of 10 to 20% may give the lowest particle size emulsions, but protein concentrations of 0.5 to 7%, particularly 0.75 to 2.5%, effectively stabilize silicone oil-in-water emulsions at median particle size in the range 1 to 30 μm.

The silicone oil-in-water emulsion stabilized by a protein or peptide may in general be produced by mixing the water, the silicone oil and the protein or peptide under high shear. Conveniently the protein or peptide may be dissolved in the water before mixing with the silicone oil. The high shear mixing apparatus may be any of those known for silicone oil-in-water production. For example the aqueous protein solution and silicone oil may be mixed in a rotor and stator mixing apparatus such as an UltraTurrax™. Further mixing may be carried out if required in an apparatus applying increased shear to give a lower particle size emulsion, for example in a homogeniser, particularly a two stage pressure homogeniser such as a Rannie™ homogeniser, or microfluidiser, or a sonolator (ultrasonic mixer).

When preparing emulsions containing a high proportion of silicone oil phase, for example above 60% wt silicone, the silicone may be mixed with protein or peptide and a small amount of water under high mechanical shear to form a non-Newtonian “thick phase”, which has a very high viscosity at low shear rates (much more viscous at low shear rate than the silicone polymer alone). The high shear mixing in this case is carried out in a mixer designed to deal with thick pastes such as a dental mixer. On continued mixing the “thick phase” converts to an oil-in-water emulsion, which may be diluted with further water, optionally containing further protein or peptide, if required.

Emulsification may be carried out batchwise or continuously, for example the aqueous protein solution and silicone oil may be fed to a continuous emulsification apparatus such as that described in WO-02/42360-A2. In any mixing apparatus the mixing tool is alternatively submerged in the aqueous silicone oil mixture to minimize entrapment of air and foam formation.

Emulsification is carried out at a temperature in the range 0 to 60° C., alternatively 15 to 50° C. The emulsion should not be heated at over 60° C. during its production as high temperatures may denature the protein, that is irreversibly coagulate the protein.

The particle size of the emulsion depends on many factors such as the amount and type of protein emulsifier, the amount and type of silicone oil and the degree of shear applied during mixing. The median size of the silicone oil droplets in the emulsion is generally≧0.2 μm, alternatively≧0.4 μm, and may be≧100 μm, alternatively≧70 μm and usually≧30 or 50 μm.

The silicone oil-in-water emulsion stabilized by a protein or peptide may contain a biocide, particularly a bactericide, to avoid bacterial growth in the emulsion. Bacterial growth may cause mould formation and may also cause degeneration of the protein or peptide which may reduce the long term stability of the emulsion. One example of a suitable bactericide is Glycacil L™ (by Lonza). Bactericide may for example be present at 0.01 to 0.25% by weight of the emulsion.

Generally the emulsion contains no non-polymeric amphiphilic surfactant. Such conventional surfactants may compete with the protein emulsifier in an unfavourable manner. If any non-polymeric amphiphilic surfactant is present in the emulsion, it is alternatively present≧25% by weight, alternatively≧10%, based on the weight of protein or peptide, and≧0.1% by weight, alternatively≧0.02%, based on the weight of the emulsion.

The silicone oil-in-water emulsion stabilized by a protein or peptide may contain one or more other additives known in silicone oil-in water emulsions, provided that the additive does not interact unfavourably with the protein or peptide. Solid additives may be present in minor amount; for example a fine hydrophobic silica may be mixed with a polydiorganosiloxane fluid before emulsifying when forming a silicone antifoam emulsion. Other additives which may be present include UV stabilizers, antioxidants, fragrances, emollients or pharmaceutical or cosmetic active materials. The other additives may be present in an amount of 0 to 30% by weight, based on the weight of the emulsion.

The silicone oil-in-water emulsion comprises 3 to 90% by weight silicone oil phase, alternatively from 5 to 75%, alternatively from 10 to 60%; and is stabilized by a protein or peptide present at 0.25 to 10% by weight of the emulsion alternatively from 0.5 to 8%, alternatively from 1 to 6%, the remainder being water and other additives. Water may be present between from 1 to 96.75%, alternatively from 10 to 75%, alternatively from 10 to 50%.

The definition of “home and/or personal care ingredients” are those ingredients that are useful in the field of home and/or personal care. The ingredients involved are in particular the ingredients used for the care of the skin, such as moisturizing, cleaning, deep cleansing, tightening, toning, skin lightening, protective (in particular protective against ultraviolet radiation or other aggressive factors, such as the cold and atmospheric pollution), anti-aging (in particular anti-wrinkle and/or firming) and slimming cares; for the care of the hair, such as conditioning, coloring, straightening, volumizing, shine; for the care of fabric, such as softness, color maintenance, ease of ironing, anti-wrinkle, water absorbancy, non yellowing, fast drying.

Skin care ingredients include emollients, moisturizers, colorants, dyes, UV absorbers and sunscreens, antiperspirants, antioxidants, fragrances, antimicrobial or antibacterial or antifungal agents, pigments, preservatives, pH controlling agents, electrolytes, chelating agents, vegetable or botanical extracts, sebum absorbants or sebum control agents, vitamins and/or moisturizers, waxes, surface active materials such as surfactants or detergents or emulsifiers, thickeners and a cosmetically acceptable medium, such as water or mixtures thereof.

Hair care ingredients include colorants, dyes, UV absorbers, preservatives, vegetable extracts, fatty alcohols, vitamins, fragrance, anti-dandruff agents, colour care additives, pearlising agents, pH controlling agents, electrolytes, chelating agents, styling agents, ceramides, amino-acid derivatives, suspending agents, surface active materials such as surfactants or detergents, thickeners and a cosmetically acceptable medium, such as water or mixtures thereof.

Home care ingredients include colorants, preservatives, fragrance, antifoam compounds, antibacterial or antifungal agents, abrasives, enzymes, optical brighteners, colour care additives, dyes transfer inhibitors, alcohols, hydrotropic agents, dye sequestrants, chelating agents, colour fixatives, anti redeposition agents, pH controlling agents, electrolytes, bleaching agents, softeners, fibres and care agents, surface active materials such as surfactants or detergents, thickeners and a medium such as water or mixtures thereof.

Examples of emollients include volatile or non-volatile silicone oils; silicone resins such as polypropylsilsesquioxane and phenyl trimethicone; silicone elastomers such as dimethicone crosspolymer; alkylmethylsiloxanes such as C30-45 Alkyl Methicone; volatile or non-volatile hydrocarbon compounds, such as squalene, paraffin oils, petrolatum oils and naphthalene oils; hydrogenated or partially hydrogenated polyisobutene; isoeicosane; squalane; isoparaffin; isododecane; isodecane or isohexa- decane; branched C8-Ci6 esters; isohexyl neopentanoate; ester oils such as isononyl isononanoate, cetostearyl octanoate, isopropyl myristate, palmitate derivatives, stearates derivatives, isostearyl isostearate and the heptanoates, octanoates, decanoates or ricinoleates of alcohols or of polyalcohols, or mixtures thereof; hydrocarbon oils of plant origin, such as wheatgerm, sunflower, grapeseed, castor, shea, avocado, olive, soybean, sweet almond, palm, rapeseed, cotton seed, hazelnut, macadamia, jojoba, blackcurrant, evening primrose; or triglycerides of caprylic/capric acids; higher fatty acids, such as oleic acid, linoleic acid or linolenic acid.

Example of waxes include hydrocarbon waxes such as beeswax, lanolin wax, rice wax, carnauba wax, candelilla wax, microcrystalline waxes, paraffins, ozokerite, polyethylene waxes.

Examples of moisturizers include lower molecular weight aliphatic diols such as propylene glycol and butylene glycol; polyols such as glycerine and sorbitol; and polyoxyethylene polymers such as polyethylene glycol 200; hyaluronic acid and its derivatives.

Examples of surface active materials or emulsifiers may be anionic, cationic or non ionic, and include organomodified silicones such as dimethicone copolyol; oxyethylenated and/or oxypropylenated ethers of glycerol; oxyethylenated and/or oxypropylenated ethers of fatty alcohols such as ceteareth-30, C12-15 pareth-7; fatty acid esters of polyethylene glycol such as PEG-50 stearate, PEG-40 monostearate; saccharide esters and ethers, such as sucrose stearate, sucrose cocoate and sorbitan stearate, and mixtures thereof; phosphoric esters and salts thereof, such as DEA oleth-10 phosphate; sulphosuccinates such as disodium PEG-5 citrate lauryl sulphosuccinate and disodium ricinoleamido MEA sulphosuccinate; alkyl ether sulphates, such as sodium lauryl ether sulphate; isethionates; betaine derivatives.

Examples of sebum absorbants or sebum control agents include silica silylate, silica dimethyl silylate, dimethicone/vinyl dimethicone crosspolymer, polymethyl methacrylate, cross-linked methylmethacrylate and aluminum starch octenylsuccinate.

Examples of vegetable or botanical extracts are derived from plants (herbs, roots, flowers, fruits, or seeds) in oil or water soluble form, such as coconut, green tea, white tea, black tea, horsetail, sunflower, wheat germ, olive, grape, pomegranate, apricot, carrot, tomato, tobacco, bean, potato, actzuki bean, catechu, orange, cucumber, avocado, watermelon, banana, lemon or palm. Examples of herbal extracts include dill, horseradish, oats, neem, beet, broccoli, tea, pumpkin, soybean, barley, walnut, flax, ginseng, poppy, avocado, pea or sesame.

Examples of pigments and colorants include surface treated or untreated iron oxides, surface treated or untreated titanium dioxide, surface treated or untreated mica, silver oxide, silicates, chromium oxides, carotenoids, chlorophyllin derivatives and yellow ocher.

Examples of UV absorbers and sunscreens include those which absorb ultraviolet light between about 290-320 nanometers (the UV-B region) such as, but not exclusively, para-aminobenzoic acid derivatives and cinnamates such as ethyulhexyol methoxycinnamate and those which absorb ultraviolet light in the range of 320-400 nanometers (the UV-A region) such as benzophenones and butyl methoxy dibenzoylmethane. Some additional examples of UV absorbers and sunscreens are 2-ethoxyethyl p-methoxycinnamate; menthyl anthranilate; homomenthyl salicylate; glyceryl p-aminobenzoate; isobutyl p-aminobenzoate; isoamyl p-dimethylaminobenzoate; 2-hydroxy-4-methoxybenzophenones sulfonic acid; 2,2′-dihydroxy-4-methoxybenzophenone; 2-hydroxy-4-methoxybenzophenone; 4-mono and 4-bis(3-hydroxy-propyl)amino isomers of ethyl benzoate; and 2-ethylhexyl p-dimethylaminobenzoate, titanium dioxide, zinc oxide, octocrylene, ethylhexyl salicylate.

Examples of thickeners include acrylamide copolymers, acrylate copolymers and salts thereof, xanthan gum and derivatives, cellulose gum and cellulose derivatives, carbomer, cassia gum, guar gum, cocamide derivatives, alkyl alcohols, gelatin, PEG- derivatives.

Examples of silicone conditioning agents for hair include silicone oils; silicone gums and mixtures or emulsions thereof; organomodified silicone oils, such as amodimethicone, aminopropyl phenyl trimethicone, phenyl trimethicone, trimethyl pentaphenyl trisiloxane, silicone quaternium-16/glycidoxy dimethicone crosspolymer, silicone quaternium-16 and mixtures or emulsions thereof.

Examples of cationic conditioning agents include guar derivatives, quaternary nitrogen derivatives of cellulose ethers; homopolymers of dimethyldiallyl ammonium chloride; copolymers of acrylamide and dimethyldiallyl ammonium chloride; homopolymers or copolymers derived from acrylic acid or methacrylic acid which contain cationic nitrogen functional groups attached to the polymer by ester or amide linkages; polycondensation products of N,N′-bis-(2,3-epoxypropyl)-piperazine or piperazine-bis-acrylamide and piperazine; and copolymers of vinylpyrrolidone and acrylic acid esters with quaternary nitrogen functionality. Specific materials include the various polyquats Polyquaternium-7, Polyquaternium-8, Polyquaternium-10, Polyquaternium-11, and Polyquaternium-23. Other categories of conditioners include cationic surfactants such as cetyl trimethylammonium chloride, cetyl trimethylammonium bromide, and stearyltrimethylammonium chloride.

Example of preservatives and cosmetic biocides include paraben derivatives, hydantoin derivatives, chlorhexidine and its derivatives, imidazolidinyl urea, phenoxyethanol, silver derivatives, salicylate derivatives, triclosan, zinc pyrithione and mixtures thereof.

Examples of antidandruff agents include pyridinethione salts, selenium compounds such as selenium disulfide, and soluble antidandruff agents.

Examples of vitamins include a variety of different organic compounds such as alcohols, acids, sterols, and quinones. They can be classified into two solubility groups: lipid-soluble vitamins and water-soluble vitamins. Lipid-soluble vitamins that have utility in personal care formulations include retinol (vitamin A), ergocalciferol (vitamin D2), cholecalciferol (vitamin D3), phytonadione (vitamin K1), and tocopherol (vitamin E). Water-soluble vitamins that have utility in personal care formulations include ascorbic acid (vitamin C), thiamin (vitamin B1) niacin (nicotinic acid), niacinamide (vitamin B3), riboflavin (vitamin B2), pantothenic acid (vitamin B5), biotin, folic acid, pyridoxine (vitamin B6), and cyanocobalamin (vitamin B 12). Additional examples of vitamins include derivatives of vitamins such as retinyl palmitate (vitamin A palmitate), retinyl acetate (vitamin A acetate), retinyl linoleate (vitamin A linoleate), and retinyl propionate (vitamin A propionate), tocopheryl acetate (vitamin E acetate), tocopheryl linoleate (vitamin E linoleate), tocopheryl succinate (vitamin E succinate), tocophereth-5, tocophereth-10, tocophereth-12, tocophereth-18, tocophereth-50 (ethoxylated vitamin E derivatives), PPG-2 tocophereth-5, PPG-5 tocophereth-2, PPG-10 tocophereth-30, PPG-20 tocophereth-50, PPG-30 tocophereth-70, PPG-70 tocophereth-100 (propoxylated and ethoxylated vitamin E derivatives), sodium tocopheryl phosphate, ascorbyl palmitate, ascorbyl dipalmitate, ascorbyl glucoside, ascorbyl tetraisopalmitate, tetrahexadecyl ascorbate, ascorbyl tocopheryl maleate, potassium ascorbyl tocopheryl phosphate or tocopheryl nicotinate.

Hair care compositions may additionally contain proteins or amino-acids, like those extracted from wheat, soy, rice, corn, keratin, elastin or silk. Most proteins are in the hydrolyzed form and they may also be quaternized.

Examples of fragrances or perfume include hexyl cinnamic aldehyde; anisaldehyde; methyl-2-n-hexyl-3-oxo-cyclopentane carboxylate; dodecalactone gamma; methylphenylcarbinyl acetate; 4-acetyl-6-tert-butyl-1,1-dimethyl indane; patchouli; olibanum resinoid; labdanum; vetivert; copaiba balsam; fir balsam; 4-(4-hydroxy-4- methyl pentyl)-3-cyclohexene-1-carboxaldehyde; methyl anthranilate; geraniol; geranyl acetate; linalool; citronellol; terpinyl acetate; benzyl salicylate; 2-methyl-3-(p-isopropylphenyl)-propanal; phenoxyethyl isobutyrate; cedryl acetal; aubepine; musk fragrances; macrocyclic ketones; macrolactone musk fragrances; ethylene brassylate.

Examples of pH controlling agents include any water soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, and citric acid.

Examples of optical brighteners or fluorescent whitening agents include stilbene derivatives, distyrylbiphenyl derivatives, coumarines, imidazolines, diazoles, triazoles and benzoxazolines.

Examples of bleaching agents include sodium perborate, sodium percarbonate and tetraacetylethylenediamine.

Examples of softeners include esterified quaternary ammonium compounds (also esterquats).

Examples of anti-redeposition agents include carboxy- methylcellulose (CMC); examples of dye transfer inhibitor include polyvinylpyrollidone.

Examples of enzymes include proteases, amylases, lipases and cellulases.

Further materials suitable for the home and/or personal care are well known to the person skilled in the art and are described in many text books as well as other publications.

Home and personal care compositions according to the invention include skin care compositions, hair care compositions and home care compositions, either rinsed or non-rinsed. Skin care compositions include shower gels, soaps, hydrogels, creams, lotions and balms and may be in the form of water-in-oil emulsion, oil-in-water emulsion, water-in-silicone emulsion, silicone-in-water emulsion or multiple emulsions such as water-in-oil-in-water or oil-in-water-in-oil. Hair care compositions include shampoos, rinse off conditioners, leave-in conditioners, gels, pomades and cuticle coats. Where hair care compositions are under the form of emulsions, they may be water-in-oil emulsion, oil-in-water emulsion, water-in-silicone emulsion, silicone-in-water emulsion. Home care compositions include liquid detergents, solid detergents, fabric softeners and hard surface cleaners. Particular home care compositions of interest are fabric care compositions.

The amount of silicone oil-in-water emulsion stabilized by a protein or peptide present in compositions according to the invention will be determined by the particular benefit to be obtained, for example, conditioning of the hair, skin or fabric. The particular level appropriate in different compositions according to the present invention is influenced by the particular composition into which it is formulated. The general level of silicone oil-in-water emulsion stabilized by a protein or peptide in the compositions may vary from 0.01 to 10% by weight, alternatively 0.05 to 2%.

At least one home and/or personal care ingredient is present from 0.01 to 99.9% wt of the final composition. The exact amount of each ingredient in the final composition is a function of its nature and its purpose and is easily determined by the man skilled in the art. For example, a skin care lotion may comprise from 5 to 95% of emollient, from 0.01 to 5% ethylhexylmethoxy cinnamate, from 0.001 to 2% vitamin, from 10 to 90% of water and from 0.5 to 5% emulsifier, based on the weight of the final composition. A hair conditioner may comprise from 0.001 to 5% cationic guar derivative, from 0.01 to 10% silicone oil, from 0.1 to 5% fatty alcohol and from 50 to 95% water based on the weight of the final composition. Generally, 0.01-5% wt preservative is suggested in personal care compositions. A fabric softener may comprise from 25 to 90% water, from 0.001 to 25% fragrance, from 1 to 10% betaine ester derivative and from 1 to 40% esterified quaternary ammonium compounds, based on the weight of the final composition.

The home and personal care compositions according to the invention are prepared by mixing the silicone oil-in-water emulsion stabilized by a protein or peptide in the aqueous phase and further combining the other compatible aqueous phase ingredients to form the aqueous phase of the composition. An oil phase may be present in the composition, and may be combined by either mixing or mixing and heating all ingredients together, or as generally known by the man skilled in the art. The emulsifiers may be added to the appropriate phase, and the oil and aqueous phase may then mixed together to form the final composition. The composition may be adjusted for pH. Sensitive ingredients may further be added as appropriate, such as fragrances, nacres. The emulsifiers may not disrupt the silicone oil-in-water emulsion stabilized by a protein or peptide. Mixing devices are those generally used by the man skilled in the art to prepare home and personal care compositions and include mixing vessels with paddles, stirrers, homogenisers, scrapers and other equipment which is known to the person skilled in the art. The process may be performed at temperatures ranging from 15 to 60° C., alternatively at room temperature (25° C.). The aqueous phase or composition containing the silicone oil-in-water emulsion stabilized by a protein or peptide should not be heated at over 60° C.

The application of the home and personal care compositions according to the invention generally generate conditioning benefit of the substrate, skin, hair, surface or fabric. Benefits obtained from using the hair care compositions according to the invention include one or more of the following benefits: hair conditioning, softness, detangling ease. Benefits obtained from using the skin care compositions according to the invention include one or more of the following benefits: skin softness, suppleness. Benefits obtained from using the home care compositions according to the invention include one or more of the following benefits: fabric softening, ease of ironing, colour care, anti-wrinkle.

The invention also comprises a method of treating a hair, skin or fabric substrate by applying to it a composition according to the first aspect of the invention.

The invention is illustrated by the following examples, in which parts and percentages are by weight and viscosities are measured at 25° C., unless otherwise indicated. These examples are intended to illustrate the invention to one of ordinary skill in the art and should not be interpreted as limiting the scope of the invention set forth in the claims.

### EXAMPLES

The following examples are included to demonstrate embodiments of the invention.

It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. All percentages are in wt. %. Stability is measured using Brookfield rheometer at weekly and monthly intervals, up to 12 months, at 25° C. Stability is defined as a constant viscosity over a certain time.

### Examples 1 to 3

### Emulsions 1 to 3

Emulsion 1 contains 50% of a mixture of cyclopentasiloxane and dimethiconol, stabilized by 2% of sodium caseinate, in water at 47.7% preserved with 0.3% of DMDM Hydantoin and iodopropynyl butylcarbamate.

Emulsion 2 contains 50% of a mixture of dimethicone and dimethiconol, stabilized by 2% of sodium caseinate, in water at 47.7% preserved with 0.3% of DMDM Hydantoin and iodopropynyl butylcarbamate.

Emulsion 3 contains 30% of a mixture of dimethicone and amodimethicone, stabilized by 3.1% of sodium caseinate, in water at 66.75% preserved with 0.13% of DMDM Hydantoin and iodopropynyl butylcarbamate.

### Examples 4 to 7

Gel compositions were prepared comprising Emulsions 1 and 2, as listed in Table 1. Examples 4 and 5 are stable for at least 12 months at room temperature, Examples 6 and 7 have decreasing viscosity after 12 months at room temperature.

Procedure to prepare gel compositions of Table 1:

Mix the thickening agent and water until homogeneous

Add Emulsion and pursue mixing until complete homogeneity

TABLE 1

| | Exam- | Exam- | Exam- | Exam- |
|---|---|---|---|---|
| Ingredients (% wt) | ple 4 | ple 5 | ple 6 | ple 7 |
| Emulsion 1 | 3 | — | 3 | — |
| Emulsion 2 | — | 3 | — | 3 |
| Sodium polyacrylate (and) | 3 | 3 | — | — |
| dimethicone (and) | | | | |
| cyclopentasiloxane (and) | | | | |
| trideceth-6 (and) PEG/PPG- | | | | |
| 18/18 dimethicone | | | | |
| Polyacrylamide (and) C13- | — | — | 2 | 2 |
| 14 isoparaffin (and) laureth-7 | | | | |
| Water | 93.7 | 93.7 | 94.7 | 94.7 |
| DMDM Hydantoin (and) | 0.3 | 0.3 | 0.3 | 0.3 |
| iodopropynyl butylcarbamate | | | | |

### Examples 8 and 9

Oil-in-water cream compositions were prepared as described in Table 2. The compositions are homogeneous and stable for at least 12 months at room temperature.

Procedure to prepare O/W compositions of Table 2:

Mix phase A ingredients until homogeneous

Mix phase B ingredients together and add phase A with mixing

Add phase C and mix until homogeneous

TABLE 2

| Ingredients (% wt) | Example 8 | Example 9 |
|---|---|---|
| Phase A | | |
| Dimethicone | 5 | 5 |
| Dimethicone (and) dimethicone crosspolymer | 25 | 25 |
| Dimethicone (and) trisiloxane | 10 | 10 |
| Caprylic/capric triglyceride | 6 | 6 |
| Sodium polyacrylate (and) dimethicone (and) | 4 | 4 |
| cyclopentasiloxane (and) trideceth-6 (and) | | |
| PEG/PPG- 18/18 dimethicone | | |
| Phase B | | |
| Water | 41.7 | 41.7 |
| Butylene Glycol | 3 | 3 |
| DMDM Hydantoin (and) iodopropynyl | 0.3 | 0.3 |
| butylcarbamate | | |
| Phase C | | |
| Emulsion 1 | 5 | — |
| Emulsion 2 | — | 5 |

### Examples 10 and 11

Water-in-silicone cream compositions were prepared as described in Table 3. The compositions are homogeneous and stable for at least 11 months at room temperature.

Procedure to prepare W/Si compositions of Table 3:

Mix phase A ingredients until homogeneous

Mix phase B ingredients until homogeneous

Add slowly phase B to phase A (drop by drop) with continuous agitation

Homogenize using high shear device (Silverson)

TABLE 3

| Ingredients (% wt) | Example 10 | Example 11 |
|---|---|---|
| Phase A | | |
| Cyclopentasiloxane (and) | 10 | 10 |
| PEG/PPG-18/18 dimethicone | | |
| Cyclopentasiloxane | 16.3 | 16.3 |
| Phase B | | |
| Emulsion 1 | 4.6 | — |
| Emulsion 2 | — | 4.6 |
| Sodium chloride | 2 | 2 |
| Water | qs | qs |

### Examples 12 and 13

Cold mix lotion compositions were prepared as described in Table 4. The compositions are homogeneous.

I Procedure to prepare cold mix lotion compositions of Table 4:

Mix phase B ingredients together

Add phase A to phase B

Mix until homogenous

TABLE 4

| Ingredients (% wt) | Example 12 | Example 13 |
|---|---|---|
| Phase A | | |
| Emulsion 1 | 20 | — |
| Emulsion 2 | — | 20 |
| Phase B | | |
| Water | 78 | 78 |
| Xanthan gum | 1 | 1 |
| Preservative mixture | 1 | 1 |

### Example 14 and Comparative Examples 1 and 2

Hydrogel compositions were prepared as described in Table 5. The compositions are stable for at least 12 months at room temperature. Sensory analyses were carried out using an experienced panel, comparing Example 14 (containing Emulsion 1) to Comparative example 1 (containing the neat cyclopentasiloxane and dimethiconol mixture) and to Comparative example 2 (containing a non ionic emulsion from synthetic surfactants of the cyclopentasiloxane and dimethiconol mixture). Panelists found that Example 14, containing the protein emulsion, gives less wetness before absorption and less greasiness after absorption than Comparative example 1 (significant differences at 95% confidence level).

Panelists found no difference before absorption between Example 14 and Comparative example 2. After absorption, Example 14 was significantly less greasy (with 95% confidence level).

Procedure to prepare hydrogel compositions of Table 5:

Disperse Carbomer in water and neutralize with triethanolamine

Add cyclopentasiloxane per small amount and mix until homogeneous

Add protein emulsion and mix until homogeneous

Add preservative mixture and mix until homogeneous

TABLE 5

| | Comparative | Comparative | Example |
|---|---|---|---|
| Ingredients (% wt) | example 1 | example 2 | 14 |
| Carbomer | 0.6 | 0.6 | 0.6 |
| Water | 81.1 | 80.15 | 76.1 |
| Triethanolamine | qs | qs | qs |
| Cyclopentasiloxane (and) | 5 | — | — |
| dimethiconol | | | |
| Cyclopentasiloxane (and) | — | 5.95 | — |
| dimethiconol (and) laureth-23 | | | |
| (and) laureth-4 | | | |
| Emulsion 1 | — | — | 10 |
| Cyclopentasiloxane | 13 | 13 | 13 |
| DMDM Hydantoin (and) | 0.3 | 0.3 | 0.3 |
| iodopropynyl butylcarbamate | | | |

### Examples 15 to 18

Sun care compositions were prepared as described in Table 6. The compositions are homogeneous and stable for at least 1 month at room temperature.

Procedure to prepare sun care compositions of Table 6:

Mix ingredients of Phase A (except zinc oxide mixture)

Mix ingredients of phase B together

Before emulsification, add zinc oxide mixture to phase A and mix until homogeneous

Slowly add phase B to phase A with turbulent mixing

Add phase C with mixing

Continue mixing for an additional 15 min

TABLE 6

| | Exam- | Exam- | Exam- | Exam- |
|---|---|---|---|---|
| Ingredients (% wt) | ple 15 | ple 16 | ple 17 | ple 18 |
| Phase A | | | | |
| Lauryl PEG/PPG- 18/18 | 3.0 | 3.0 | — | — |
| Methicone | | | | |
| Bis-Isobutyl PEG/PPG- 10/7 | — | — | 3.0 | 3.0 |
| Dimethicone Copolymer | | | | |
| Dimethicone (and) trisiloxane | 16.0 | 16.0 | 16.0 | 16.0 |
| Octyl Methoxycinnamate | 7.0 | 7.0 | 7.0 | 7.0 |
| Zinc oxide (and) dimethicone | 5.0 | 5.0 | 5.0 | 5.0 |
| Phase B | | | | |
| Sodium chloride | 2.0 | 2.0 | 2.0 | 2.0 |
| Polysorbate 20 | 0.4 | 0.4 | 0.4 | 0.4 |
| Water | 62.1 | 62.1 | 62.1 | 62.1 |
| Emulsion 1 | 4.0 | — | 4.0 | — |
| Emulsion 2 | — | 4.0 | — | 4.0 |
| Phase C | | | | |
| Propylene glycol (and) | 0.5 | 0.5 | 0.5 | 0.5 |
| diazolidinyl urea (and) | | | | |
| iodopropynyl butylcarbamate | | | | |

### Examples 19 and 20

Roll on anti-perspirant compositions were prepared as described in Table 7. The compositions are homogeneous and stable for at least 9 months at room temperature.

Procedure to prepare roll on anti-perspirant compositions of Table 7:

Mix ingredients of phase A together

Mix ingredients of phase B together

Separately heat phase A and phase B to 70° C.

Add phase B to phase A with mixing

Cool to 50° C.

Mix ingredients of phase C together

Add phase C to the previous mix

Cool to room temperature

Mix ingredients of Phase D together

Add phase D with slow mixing

TABLE 7

| Ingredients (% wt) | Example 19 | Example 20 |
|---|---|---|
| Phase A | | |
| Water | 41 | 41 |
| Steareth-20 | 0.6 | 0.6 |
| Phase B | | |
| Behentrimonium methosulfate | 1 | 1 |
| (and) cetearyl alcohol | | |
| Steareth-2 | 1.4 | 1.4 |
| PPG-3 Myristyl Ether | 3 | 3 |
| Phase C | | |
| Water | 15 | 15 |
| Aluminium Zirconium | 30 | 30 |
| Tetrachlorohydrex GLY | | |
| Phase D | | |
| Emulsion 1 | 3 | 0 |
| Emulsion 2 | 0 | 3 |
| Water | 5 | 5 |

### Example 21

A rinse off conditioner composition was prepared as described in Table 8.

Procedure to prepare rinse off conditioner composition of Table 8:

Heat phase A water to 75° C. and add hydroxyethylcellulose under high speed mixing

Melt phase B ingredients at 80° C.

Heat phase C to 80° C.

Add phase C to phase B

Slowly add phase A to phase B+C

Let cool down to room temperature while gently mixing

Add Emulsion 3 under mixing

Add the preservative

Adjust pH to 4 by adding citric acid solution at 1%

TABLE 8

| Ingredients (% wt) | Example 21 |
|---|---|
| Phase A | |
| Water | 47.49 |
| Hydroxyethylcellulose | 1.42 |
| Phase B | |
| Glyceryl Stearate (and) PEG-100 stearate | 0.95 |
| Cetyl Alcohol | 0.95 |
| Phase C | |
| Water | 38.47 |
| Phase D | |
| Emulsion 3 | 4.74 |
| DMDM Hydantoin | 0.49 |
| Citric Acid 1% | 5.49 |

### Example 22

A leave in conditioner composition was prepared as described in Table 9.

Procedure to prepare leave in conditioner compositions of Table 9:

Heat water to 75° C. and add EDTA

Add methyl paraben

Cool to 50° C.

Mix Phase C

Add phase B with mixing

Cool to 40° C.

Add phase C and mix until uniform

Compensate for water loss

Adjust pH to 4.5-5 with citric acid

TABLE 9

| Ingredients (% wt) | Example 22 |
|---|---|
| Phase A | |
| Water | 81.80 |
| EDTA | 0.20 |
| Methyl Paraben | 0.10 |
| Phase B | |
| Polyquaternium-10 | 0.50 |
| Panthenol | 0.20 |
| DMDM Hydantoin | 0.20 |
| Glycerin | 11.00 |
| Hydrolyzed Partially hydrolysed wheat | 1.00 |
| protein Gluten | |
| Phase C | |
| PEG-12 Dimethicone | 1.00 |
| Emulsion 3 | 4.00 |
| Phase D | |
| Citric acid 1% | qs |

### Examples 23 to 29

### Emulsions 4 to 8 and Comparative Examples Ref1 to Ref3

Silicone oil-in-water emulsions stabilized by a protein or peptide were also used in rince cycle softener applications as follows.

TABLE 10

| | Silicone mixture | |
|---|---|---|
| Emulsions | (50% of final emulsion) | Protein - 2% |
| Emulsion 4 | 8040/dimethicone - 50/50 | partially hydrolysed |
| | | wheat protein |
| Emulsion 5 | 8630/dimethicone - 50/50 | partially hydrolysed |
| | | wheat protein |
| Emulsion 6 | 8040/dimethicone - 50/50 | sodium caseinate |
| Emulsion 7 | 8040/dimethicone - 25/75 | partially hydrolysed |
| | | wheat protein |
| Emulsion 8 | 8040/dimethicone - 75/25 | partially hydrolysed |
| | | wheat protein |

References

Ref1=neat rinse cycle softener base

Ref2=rinse cycle softener base+3% of cationic emulsion with synthetic surfactants of slightly crosslinked silicone polymer

Ref3=commercial two-in-one detergent

Rinse cycle softener compositions or liquid detergent compositions were prepared by adding 3% of test Emulsion—described in Table 10—to a base. The Emulsion containing compositions were compared to either the references or between each other by a panel of 16 volunteers, who ranked the towels treated with the products for their softness and gave them a score compared to the reference towel.

The rinse cycle softener base is water-based and contains 16% quaternary compounds. The mixtures were moderately stirred overnight and were used as Rinse Cycle Fabric

Softener (RCFS) no later than 24 h after their preparation.

RCFS with Emulsion 4 (Example 23) and RCFS with Emulsion 5 (Example 24) are found softer than Ref1 (13 and 10 on 16 respectively), and equivalent to each other.

RCFS with Emulsion 4 (Example 23) and RCFS with Emulsion 6 (Example 25) are found equivalent to each other.

RCFS with Emulsion 8 (Example 27) is found softer than RCFS with Emulsion 7 (Example 26) and equivalent to Ref4.

Liquid detergent containing Emulsion 4 (Example 28) and liquid detergent containing Emulsion 5 (Example 29) were found equivalent to each other and equivalent to Ref3.

1. A home and personal care composition comprising (i) a silicone oil-in-water emulsion stabilized by a protein or peptide wherein the emulsion contains no non-polymeric amphiphilic surfactant and wherein the protein does not include proteins whose natural function is as structural proteins, and (ii) at least one home and personal care ingredient suitable for care of skin, care of hair or care of fabric.

2. The home and personal care composition of claim 1 where the silicone oil-in-water emulsions stabilized by a protein or peptide comprises 3 to 90% weight silicone oil and is stabilized by a protein or peptide present at 0.25 to 10% by weight of the emulsion.

3. The home and personal care composition of claim 1 where the silicone oil-in-water emulsions stabilized by a protein or peptide (i) is present from 0.01 to 10% by weight and the at least one home and personal care ingredient (ii) is present from 0.01 to 99.9% by weight.

4. (canceled)

5. The composition according to claim 1 wherein ingredient (ii) is suitable for the care of skin.

6. The composition according to claim 1 wherein ingredient (ii) is suitable for the care of hair.

7. The composition according to claim 5 wherein the ingredient suitable for the care of skin is selected from emollients, moisturizers, colorants, dyes, UV absorbers and sunscreens, antiperspirants, antioxidants, fragrances, antimicrobial or antibacterial or antifungal agents, pigments, preservatives, pH controlling agents, electrolytes, chelating agents, vegetable or botanical extracts, sebum absorbants or sebum control agents, vitamins and/or moisturizers, waxes, surface active materials, thickeners and a cosmetically acceptable medium or mixtures thereof.

8. The composition according to claim 6 wherein the ingredient suitable for the care of hair is selected from colorants, dyes, UV absorbers, preservatives, vegetable extracts, fatty alcohols, vitamins, fragrance, anti-dandruff agents, colour care additives, pearlising agents, pH controlling agents, electrolytes, chelating agents, styling agents, ceramides, amino-acid derivatives, suspending agents, surface active materials, thickeners and a cosmetically acceptable medium or mixtures thereof.

9. The composition according to claim 1 wherein ingredient (ii) is suitable for the care of fabric.

10. The composition according to claim 9 wherein the ingredient suitable for the care of fabric is selected from colorants, preservatives, fragrance, antifoam compounds, antibacterial or antifungal agents, abrasives, enzymes, optical brighteners, colour care additives, dyes transfer inhibitors, alcohols, hydrotropic agents, dye sequestrants, chelating agents, colour fixatives, anti redeposition agents, pH controlling agents, electrolytes, bleaching agents, softeners, fibres and care agents, surface active materials, thickeners and water or mixtures thereof.

11. (canceled)

12. The composition according to claim 5, which is a shower gel, soap, hydrogel, cream, lotion or balm.

13. (canceled)

14. The composition according to claim 6, which is a shampoo, rinse off conditioner, leave-in conditioner, gel, pomade or cuticle coat.

15. The composition according to claim 9, which is a fabric care composition, a liquid detergent, a solid detergent, a fabric softener or a hard surface cleaner.

16. A method of treating a hair, skin or fabric substrate by applying to it the composition as claimed in claim 1.
